(19) United States
(12) Patent Application Publication
(10) Pub. No.: US 2003/0069476 A1
(43) Pub. Date: Apr. 10, 2003

(54) Dilating inflatable speculum

(76) Inventors: Richard J. Deslauriers, Woodbury, CT (US); Robert T. Potash, South Windsor, CT (US); Lewis W. Chappel, New Hartford, CT (US)

Correspondence Address: Richard J. Deslauriers, M.D., 50 Altair Avenue, Plymouth, CT, 06782, US

(21) Appl. No.: 09/972,254

(22) Filed: Oct. 9, 2001

(51) Int. Cl. A61B 17/02

(52) U.S. Cl. 600/207

(57) ABSTRACT

An unrolling inflatable speculum comprising an insertion device, wherein said insertion device comprises a mandrel having a shaft portion and a head portion, a baffled bladder, wherein said baffled bladder comprises an open cell structure and an at least one such that access is permitted through the surface of said bladder through said opening, and an inflation mechanism, wherein said inflation mechanism is comprised of a handheld bulb-pump and an inflation line. The baffled bladder is rolled around said shaft portion and inserted into a cavity, and wherein said inflation mechanism provides for radial expansion of said baffled bladder said cavity from a rolled position to a cylindrical shape through progressively increasing the internal pressure of said baffled bladder to a pressurized state such that a lateral and radial support of said baffled bladder is provided such that said cavity does not collapse said expanded baffled bladder and such that said cavity is expanded, and wherein said baffled bladder provides for expansion of said bladder to said cylindrical shape having a maximum internal diameter.

### BACKGROUND OF THE INVENTION

1. Field of the Invention

This invention relates to the field of vaginal speculums, and more particularly to inflatable and dilating vaginal speculums.

2. Description of the Related Art

In performing vaginal examinations, doctors and physicians may use a vaginal speculum, whereby the vaginal cavity may be dilated or expanded to permit the physician or doctor to look at the cervix and the vaginal walls in the vaginal cavity, or for the physician to insert tools into the vaginal cavity in the performance of the examination, such as taken a sample of tissue from the cavity. In order to accomplish the dilation of the cavity, a device with sufficient rigidity is required so as to prevent the vaginal cavity from collapsing around the speculum. A number of different speculum embodiments have been proposed with each, though accomplishing certain objectives, failing to address all of the concerns presented in vaginal examinations, whereby additional time in the examination may be required. Furthermore, a number of different devices may be required to perform a complete examination, whereby the speculum only provides for viewing of the vaginal cavity and another device may be required for the dilation of the vaginal cavity. Still, another shortcoming of the prior art is adequately addressing the need for patient comfort, while still meeting the objective of dilating the vaginal cavity.

One type of speculum utilizes two or more engagement blades, which upon insertion into the vaginal cavity may be activated whereby the engagement blades separate. This type of speculum performs adequately, however, poses a significant shortcoming in patient comfort as these devices are made of a rigid material usually metal or hard plastic and is not comfortable for the patient. Additionally, these devices may have a tendency to cause lacerations inside the vaginal cavity due to the sharp edges of the engagement blades. Furthermore, these devices may not provide for sufficient sidewall support of the vaginal cavity, and as such the vaginal cavity may cave in on these devices substantially increasing the difficulty in its application and increasing patient discomfort.

Other speculums teach the use of inflatable speculums, which may have the ability to increase the patient's comfort level during their application. However, a shortcoming of the prior art is that these speculums may not provide enough lateral support to prevent the vaginal cavity from collapsing the structure of the speculum. Another shortcoming is that these inflatable speculums may have a solid wall structure, and as such may not permit the examining physician the ability to examine the walls of the vaginal cavity, such that sampling may not be conducted. Still yet another shortcoming may be that these devices may have a tendency towards internal ballooning of the inflatable structure, once these devices have been inflated, and such that viewing area may be substantially reduced.

### BRIEF SUMMARY OF THE INVENTION

Therefore a need has arisen for an inflatable speculum that overcomes these and other shortcomings of the related art. One technical advantage of the present invention is that this speculum may provide for increased patient comfort from the use of inflatable technology. Another technical advantage is that this invention may provide for sufficient lateral strength that may prevent the device from collapsing on itself during its application. Yet another technical advantage of the present invention is that it may permit radial expansion of a bladder with baffles through pressurization such that ballooning of the bladder may be eliminated as to permit sufficient dilation of the vaginal walls while retaining a maximum unobstructed internal diameter such as to permit viewing of the cervix. Still yet another technical advantage is that this device may permit simultaneous examination of the vaginal cavity and the sampling of the cervical cells and from the vaginal walls by the physician.

According to an exemplary embodiment of this invention, an unrolling inflatable speculum that may comprise an insertion device, wherein said insertion device may comprise a mandrel having a shaft portion and a head portion. Furthermore, this device may comprise a baffled bladder, wherein said baffled bladder comprises an open cell structure, such that excessive ballooning may be eliminated, and further comprising an at least one opening, such that access is permitted through the inner channel to the vaginal wall through said at least one opening. Still in its exemplary embodiment, this speculum may comprise an inflation mechanism, wherein said inflation mechanism may be comprised of a handheld bulb-pump and an inflation line, such that a controlled and uniform pressurization of said bladder is accomplished.

A preferred method of use that may comprise unrolling said baffled bladder from a rolled position around a mandrel, after this device has been inserted into a cavity, and wherein said baffled bladder may be expanded in said cavity from said rolled position to a cylindrical shape through progressively increasing the internal pressure of said baffled bladder to a pressurized state, such that a lateral and radial support of said baffled bladder may be provided that may prevent said cavity from collapsing said expanded baffled bladder and such that said cavity is simultaneously expanded along with the expansion of said bladder. Additionally, said baffled bladder may provide for expansion of said bladder, such that a minimum thickness of said baffled bladder in its pressurized state is maintained, such that a maximum internal diameter permitting unobstructed viewing of the cervix without ballooning of the baffled bladder is provided.

Other objects, features, and advantages will be apparent to persons of ordinary skill in the art in view of the following detailed description of the invention and the accompanying drawings.

### BRIEF DESCRIPTION OF THE SEVERAL VIEWS

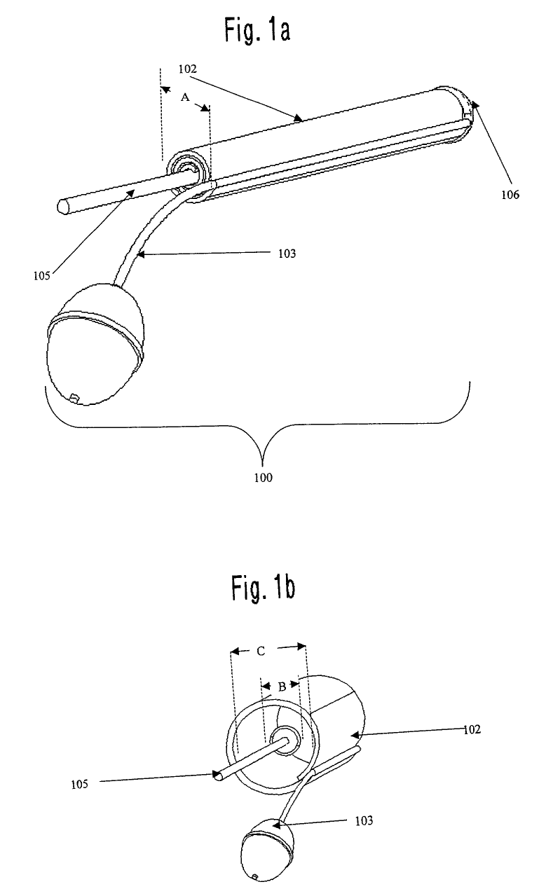

FIG. 1a depicts a speculum according to a preferred embodiment of this invention.

FIG. 1b depicts a speculum according to a preferred embodiment of this invention.

FIG. 1c depicts a speculum according to another embodiment of this invention.

FIG. 1d depicts a speculum according to another embodiment of this invention.

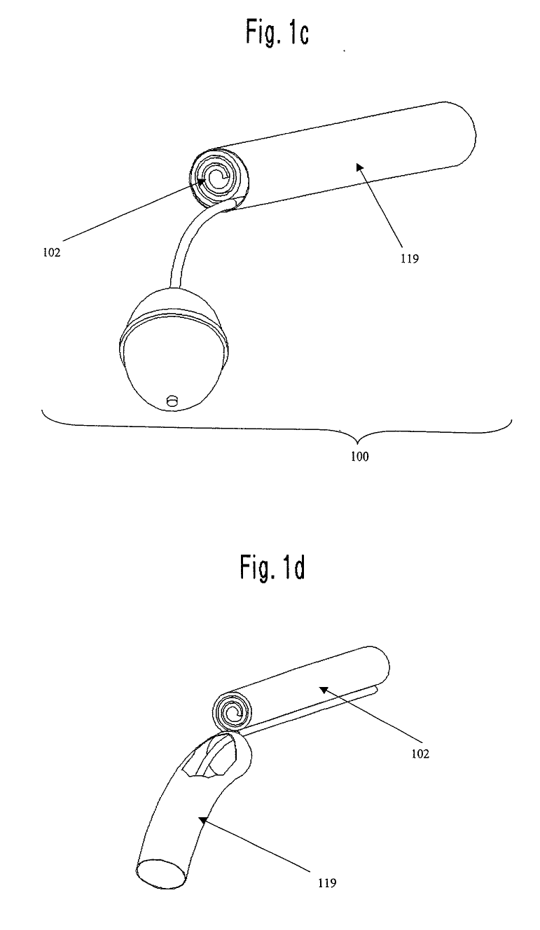

FIG. 2 depicts a bladder for a speculum according to the embodiments of this invention.

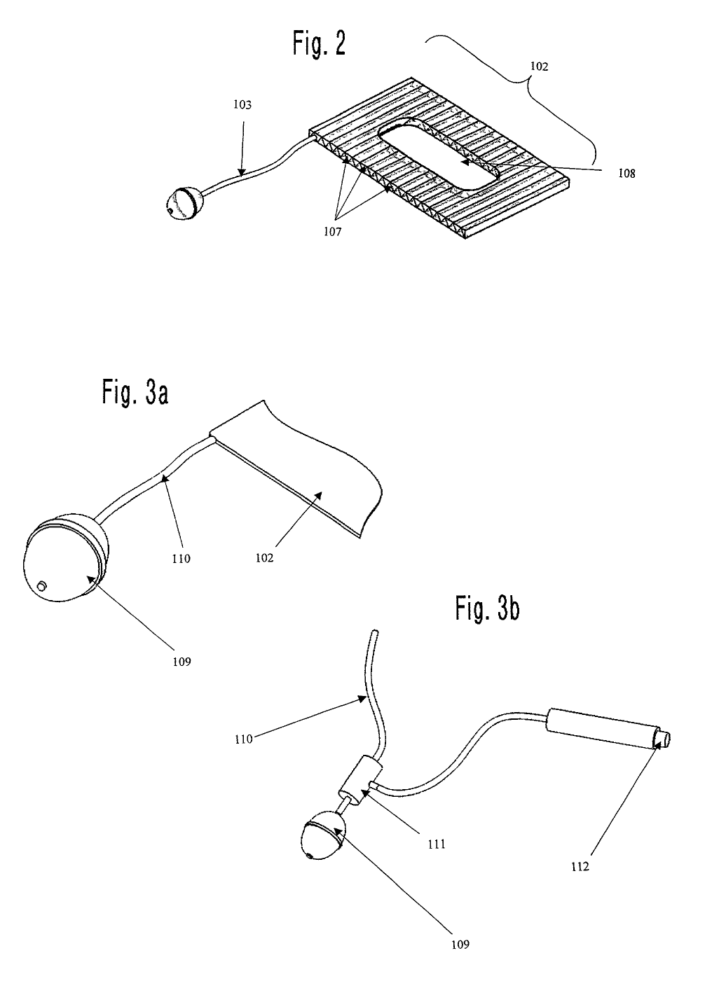

FIG. 3a depicts an inflation device for a speculum according to the embodiments of this invention.

FIG. 3b depicts a pressure release valve for a speculum according to the embodiments of this invention.

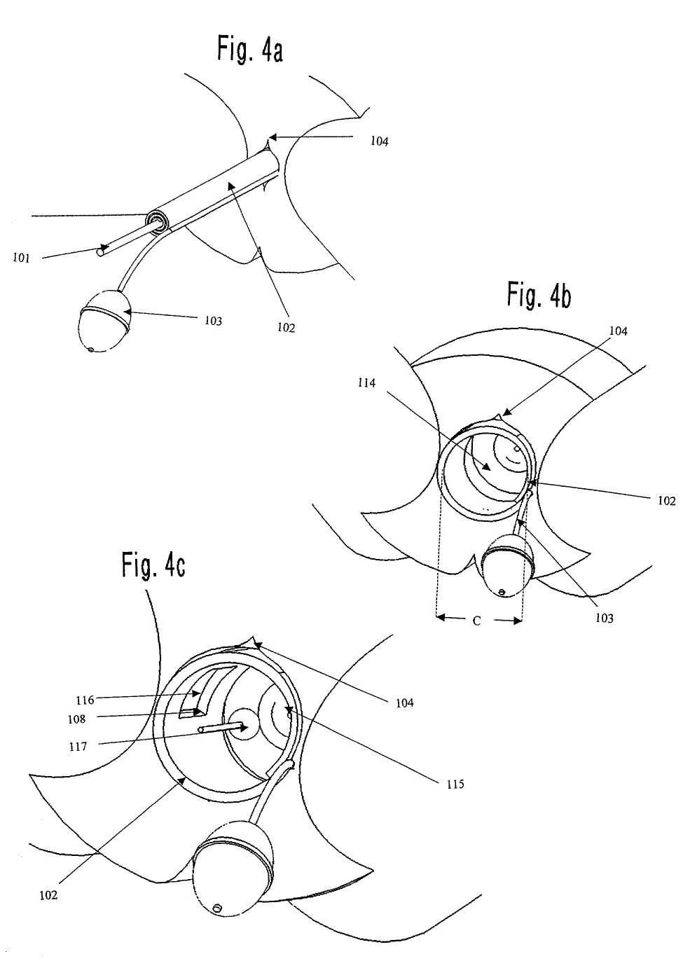

FIG. 4a depicts a method of using a speculum according to the embodiments of this invention.

FIG. 4b depicts a method of using a speculum according to the embodiments of this invention.

FIG. 4c depicts a method of using a speculum according to the embodiments of this invention.

### DETAILED DESCRIPTION OF THE INVENTION

Preferred embodiments of the present invention and their advantages may be understood by referring to FIGS. 1a-4c, like numerals being used for like corresponding parts in the various drawings.

According to FIGS. 1a and 1b, an exemplary embodiment of an unrolling inflatable speculum 100 in its rolled state is described. Speculum 100 comprises insertion mechanism 101, baffled bladder 102 and inflation mechanism 103. Specifically, insertion mechanism 101 may provide for simplified insertion of said baffled bladder 102 into a cavity 104. In an exemplary embodiment, insertion mechanism 101 may be a mandrel, wherein said mandrel may comprise a shaft portion 105 and a head portion 106. According to this embodiment bladder 102 may be rolled around shaft portion 105, such that head portion 106 at its widest may have a width B and where bladder 102 may have a width A in its rolled state, and wherein width B is larger than the width A. In this embodiment bladder 102 may remain secure to shaft portion 105 until expansion of bladder 102 in said cavity 104, such that the diameter C of bladder 102 in its unrolled, or expanded, state may be greater than the maximum width B of head portion 106, such that said mandrel may be removed through drawing said mandrel through said diameter.

FIG. 1c and FIG. 1d depict another embodiment of insertion mechanism 101. In this embodiment insertion mechanism 101 may comprise sheath 119 for enclosing said bladder 102 in a rolled position. Further, sheath 119 may comprise a smooth external surface, such that the smooth external surface may facilitate the insertion of insertion mechanism 101 into cavity 104 without causing discomfort to a patient. Furthermore in this embodiment, sheath 119 may be comprised of an elastic material, or alternatively, said sheath may be substantially inflexible, such as a plastic. Lastly, in this embodiment of speculum 100, insertion mechanism 101 may be slidably removed and disengaged from rolled bladder 102, such that sheath 119 may be removable without causing the removal of bladder 102, and such that bladder 102 may remain in its rolled state while insertion mechanism 101 is removed from bladder 102.

Referring to FIGS. 2, the preferred embodiments of bladder 102 are shown. Specifically, bladder 102 may comprise a hollow pliable material that may define a volume and a thickness. Bladder 102 may further comprise baffles 107, wherein baffles 107 may further comprise an open cell structure that may serve the function of bonding the outer surface to the inner surface, and such that baffles 107 may maintain a maximum thickness of bladder 102 in a pressurized state. Furthermore, bladder 102 in its pressurized state expands from its rolled position within cavity 104, as shown in FIGS. 4a-4c, to take the shape of cavity 104, such that where cavity 104 is cylindrical, then upon being pressurized bladder 104 takes a cylindrical shape. Additionally, bladder 102 may be comprised of a material such that, when bladder 102 is pressurized, then combined with baffles 107, bladder 102 may provide for sufficient lateral sidewall support that may resist the force being exerted by an expanding cavity 104. Furthermore, bladder 102 may comprise an at least one opening 108, such that access is permitted from the inner surface of bladder 102 through the center bladder 102.

In other embodiments, bladder 102 may comprise a biocompatible material, and may further be of a transparent nature that may permit the viewing from the inner surface through the outer surface. The biocompatible material of bladder 102 may be selected from one of the group consisting of polypropylene, polyethylene, rubber, or plastic. Furthermore in the preferred embodiments, said biocompatible material may further comprise an anti-microbial compound coated onto the surfaces of bladder 102, or alternatively, said biocompatible material may comprise an integrated anti-microbial compound, and such that the integration of an anti-microbial agent onto bladder 102 may provide for increased sterility and the reduction of the potential of causing infection within and around cavity 104.

Referring to FIG. 3a, the preferred embodiments of inflation mechanism 103 are shown. Inflation mechanism 103 may be comprised of a handheld bulb-pump 109 and an inflation line 110, wherein inflation line 110 may comprise a hollow tube that extends from pump 109 and may be connected to an at least one section of bladder 102. Alternatively in another embodiment, not shown, inflation mechanism 103 may be comprised of an electric pump and an inflation line, wherein said inflation line may comprise a hollow tube that extends from electric pump to an at least one section of bladder 102. In these embodiments, inflation mechanism 103 may provide for a means of causing the pressurization of bladder 102.

Referring to FIG. 3b, a preferred embodiment of inflation mechanism 103 may further comprise valve 111, wherein valve 111 may permit the release of pressure. In an exemplary embodiment, release valve 111 may comprise a patient engagement mechanism 112, such that a patient, who begins to experience discomfort during or after pressurization of bladder 102 within patient's cavity 104, may activate release valve 111 causing rapid depressurization of bladder 102. In other embodiments, not shown, release valve 111 may further comprise a secondary physician release valve 113, where the examiner may be provided with a means to rapidly depressurize bladder 102 upon a patient experience discomfort from the expansion of bladder 102 within cavity 104.

Referring to FIGS. 4a-4c, a method of expanding a vaginal cavity and performing a vaginal examination is shown. An inflatable speculum 100 is shown that may comprise an insertion mechanism 101, a bladder 102, and an inflation mechanism 103. The steps of the preferred method of use may comprise first inserting speculum 100 into a cavity 104, wherein said cavity may be the vaginal cavity, and wherein said insertion mechanism 101 may contain bladder 102 in a rolled position. Insertion mechanism 101 may be placed within cavity 104, and then bladder 102 may be expanded in a radial direction, such that cavity 104 may expand simultaneously along with the expansion of bladder 102.

After expansion, bladder 102 may have a cylindrical shape having an internal diameter C, and wherein insertion mechanism 101 may then be removed, such that an unobstructed center pathway 114 may be formed allowing direct viewing of the cervix 115, such that the conduction of a cervical exam may be facilitated, and which, method may further permit a sample may be taken from the cervix with swab 117 for conducting a pap smear, wherein a sample of the cervical tissue is extracted through center pathway 114. According to this preferred method, inflation mechanism 101 may provides for the expansion of bladder 102 to a cylindrical shape having an internal diameter C, and further such that a minimum thickness of said baffled bladder in its pressurized state may be maintained and not subject to inward ballooning of bladder 102, and wherein an internal diameter is maximized. Furthermore, this method may permit the viewing of vaginal walls 116, wherein said bladder may be transparent. Additionally, a sample may be extracted from the vaginal walls through opening 108 in the surface of bladder 102.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in art from a consideration of the specification or practice of the invention disclosed herein.

What is claimed is:

1. An unrolling inflatable speculum comprising:
a baffled bladder; and
an inflation mechanism,
Wherein a lateral and radial support is provided such that said baffled bladder does not collapse in a pressurized state, and wherein said baffled bladder pressurizes and expands to form a cylindrical shape having an internal diameter, such that a minimum thickness of said baffled bladder is maintained in its pressurized state, such that a maximum internal diameter permitting unobstructed viewing of the cervix without ballooning of the baffled bladder is provided, and wherein said inflation mechanism provides for radial expansion of said baffled bladder within a cavity from a rolled position to a cylindrical shape through progressively increasing the internal pressure of said baffled bladder to a pressurized state, and.

2. The speculum of claim 1, further comprising an insertion mechanism, wherein said insertion device provides for simplified insertion of said baffled bladder into a cavity.

3. The speculum of claim 2, wherein said insertion device comprises a sheath for enclosing said bladder in a rolled position.

4. The speculum of claim 3, wherein said insertion device comprises a smooth external surface.

5. The speculum of claim 3, wherein said insertion device is comprised of an elastic material.

6. The speculum of claim 3, wherein said insertion device is comprised of an inflexible material.

7. The speculum of claim 3, wherein said insertion device comprises a release mechanism.

8. The speculum of claim 2, wherein said insertion device is a mandrel, wherein said mandrel comprises a shaft portion and a head portion, and wherein said bladder is rolled around said shaft portion.

9. The speculum of claim 8, wherein said mandrel remains secured to said bladder until expansion of said bladder in said cavity, wherein said bladder diameter is of a diameter such that said mandrel may be removed through drawing said mandrel through said diameter.

10. The device of claim 1, wherein said baffled bladder comprises an open cell structure.

11. The device of claim 1, wherein said baffled bladder is comprised of a pliable biocompatible material.

12. The device of claim 11, wherein said biocompatible material is selected from one of the group consisting of polypropylene, polyethylene, rubber, or plastic.

13. The device of claim 11, wherein said biocompatible material comprises an anti-microbial compound coated onto an external surface of said baffled bladder.

14. The device of claim 11, wherein said biocompatible material comprises an integrated anti-microbial compound.

15. The device of claim 11, wherein said biocompatible material is transparent.

16. The device of claim 1, wherein said bladder comprises an at least one opening such that access is permitted through the surface of said bladder.

17. The device of claim 1, wherein said inflation mechanism is comprised of a handheld bulb-pump and an inflation line, wherein said inflation line is connected to an at least one section of said baffled bladder, and such that said inflation mechanism provides for the pressurization of said baffled bladder.

18. The speculum of claim 17, wherein said inflation mechanism further permits the release of pressurization through an emergency release valve.

19. The speculum of claim 18, wherein said emergency release valve attached to a patient control portion.

20. The speculum of claim 18, wherein said emergency release valve attached to a physician control portion.

21. The device of claim 1, wherein said inflation mechanism is comprised of an electric pump and an inflation line, wherein said inflation line is connected to an at least one section of said baffled bladder, and such that said inflation mechanism provides for the pressurization of said baffled bladder.

22. The speculum of claim 21, wherein said inflation mechanism further permits the release of pressurization through an emergency release valve.

23. The speculum of claim 21, wherein said emergency release valve attached to a patient control portion.

24. The speculum of claim 21, wherein said emergency release valve attached to a physician control portion.

25. An unrolling inflatable speculum comprising:
an insertion device, wherein said insertion device comprises a mandrel having a shaft portion and a head portion;
a baffled bladder, wherein said baffled bladder comprises an open cell structure and an at least one such that access is permitted from the inner channel to the exterior surface of said bladder through said opening; and
an inflation mechanism, wherein said inflation mechanism is comprised of a handheld bulb-pump and an inflation line,
wherein said baffled bladder is rolled around said shaft portion and inserted into a cavity, and wherein said inflation mechanism provides for radial expansion of said baffled bladder said cavity from a rolled position to a cylindrical shape through progressively increasing the internal pressure of said baffled bladder to a pressurized state such that a lateral and radial support of said baffled bladder is provided such that said cavity does not collapse said expanded baffled bladder and such that said cavity is expanded, and wherein said baffled bladder provides for expansion of said bladder to said cylindrical shape having a maximum internal diameter.

26. The speculum of claim 25, wherein said mandrel remains secured to said bladder until expansion of said bladder in said cavity, wherein said bladder diameter is of a diameter such that said mandrel may be removed through drawing said mandrel through said diameter.

27. The speculum of claim 25, wherein said inflation mechanism further permits the release of pressurization through an emergency release valve.

28. The speculum of claim 27, wherein said emergency release valve attached to a patient control portion.

29. The speculum of claim 27, wherein said emergency release valve attached to a physician control portion.

30. A method of expanding a vaginal cavity and performing a vaginal examination with the device of claim 26 comprising:
inserting said device into a vaginal cavity;
expanding said bladder radially such that said vaginal cavity is expanded simultaneously;
said mandrel is then removed such that an unobstructed center pathway is formed allowing direct viewing of the cervix and the vaginal walls.

31. The method of claim 30, wherein a direct view of the cervix is created allowing a physician to conduct a cervical examination of a patient.

32. The method of claim 30, wherein a sample of cells may be taken from the cervix for conducting a pap smear.

33. The method of claim 32, wherein a sample from the vaginal walls may taken through said at least one opening in said bladder.
